(19) United States
(12) Patent Application Publication
(10) Pub. No.: US 2026/0337642 A1
(43) Pub. Date: Oct. 29, 2026

(54) WHOLE-CANE CO-FERMENTATION OF LIGNOCELLULOSE AND SOLUBLE SUGAR TO PRODUCE ETHANOL

(71) Applicant: TERRAGIA BIOFUEL INC., Hanover, NH (US)

(72) Inventor: Lee R. LYND, Meriden, NH (US)

(21) Appl. No.: 19/103,776

(22) PCT Filed: Aug. 18, 2023
(86) PCT No.: PCT/US2023/030588

§ 371 Date: Feb. 13, 2025

Related U.S. Application Data
(60) Provisional application No. 63/399,020, filed on Aug. 18, 2022.

(51) Int. Cl. C12P 7/10

(52) U.S. Cl. CPC C12P 7/10

(57) ABSTRACT

A method for converting sugar-rich feedstocks to ethanol and other desired products is described. The method comprises co-fermentation of sugar-rich liquids and lignocellulose-rich solids to produce ethanol or other desired products.

### CROSS-REFERENCE TO RELATED APPLICATION

This application claims benefit of priority to U.S. Provisional Patent Application No. 63/399,020 filed on Aug. 18, 2022, the content of which is incorporated herein by reference in its entirety.

### BACKGROUND

### I. Field of the Invention

The disclosure relates to conversion of biomass to biofuel or other useful products. More particularly, the disclosure pertains to the co-fermentation of sugar-rich liquids with lignocellulose-rich solids.

### II. Description of the Related Art

Sugar cane and sugar beets have been used for centuries to produce sugar used as a dietary sweetener. The first step in such sugar production is milling of the feedstock followed by separation of a solids-free, sugar-rich juice from lignocellulose-rich solids. This separation is generally carried out in a large, expensive, multi-stage industrial unit operation in which flows of solids and liquid are arranged in a counter-current fashion such that the exiting liquid contains essentially all (typically on the order of 98%) of the originally present sugar, and the exiting solids are nearly sugar-free. The sugar-containing liquid is subsequently processed though multiple steps into crystallized sugar suitable for sale and into molasses. The lignocellulose-rich solids, called bagasse in the case of sugar cane processing, are typically dried and used as a process fuel although other uses have been suggested and are used to some extent (e.g., as animal feed or bedding, or as a feedstock for production of fuels or chemicals).

In the current art, solids/liquid separation is required to produce sugar because it would be unacceptable to have lignocellulose-rich solids in sugar. In the current art, Solids/liquid separation is also required and universally practiced for fermentation of sugar cane juice in the widely replicated Brazilian ethanol process, which features cell recycle that would be impaired if solids were present.

Production of biofuels, and specifically ethanol, from lignocellulose has been proposed and practiced. Biologically mediated solubilization of lignocellulose is widely thought to require thermochemical pretreatment to increase the accessibility of the feedstocks to biologically mediated solubilization.

In the case of sugar-rich feedstocks such as sugar cane, sugar beets, and energy cane (a high productivity variant on sugar cane with somewhat lower sugar content), solids-liquid separation is desired in processes featuring thermochemical pretreatment to avoid sugar degradation, which leads to decreased sugar available for biofuel production as well as compounds that inhibit fermentation. The problem is that combinations of heat and added chemicals that are sufficiently aggressive to disrupt lignocellulose tend to also degrade soluble sugars. Thus, for these sugar-rich feedstocks, current understanding and practice is that sugar-containing liquid needs to be separated from lignocellulose-rich solids prior to thermochemical pretreatment of solids and their subsequent biological conversion to biofuels.

In short, whether for sugar production, production of biofuels from sugar-rich liquid, production of biofuels from lignocellulose, or a combination of these, current practice is to separate liquid and solid components when processing sugar-rich feedstocks such as sugar cane, sugar beets, or energy cane.

Milling during fermentation (cotreatment) has been introduced as an alternative to thermochemical treatment for making lignocellulose accessible to biological attack (Balch et al., 2017 and 2020; Holwerda et al., 2019; U.S. Pat. No. 10,533,194). This approach appears to be distinctively compatible with consolidated bioprocessing using thermophilic, lignocellulose-fermenting anaerobic bacteria, since these microbes are: a) more effective at lignocellulose deconstruction than commercial fungal cellulase; and b) highly resistant to mechanical disruption, whereas yeast is not.

### SUMMARY

Disclosed is a method for converting sugar-rich feedstock into ethanol or other desired products. Substantial, potentially game-changing, cost savings are anticipated when the solids/liquid separation is not undertaken. According to the instant disclosure, sugar-rich liquid and lignocellulose-rich solids are co-fermented without being separated first, to produce biofuels. As compared to separate processing of such liquids and solids, co-fermenting them may offer further advantages by enhanced function of the viability of microbial biocatalysts.

In some embodiments, system and methods are disclosed to convert sugarcane feedstocks to ethanol using consolidated bioprocessing with cotreatment. Consolidated bioprocessing (CBP) refers to one-step biological conversion of lignocellulose without adding enzymes during the process. In one aspect, engineered thermophilic bacteria are used which have superior lignocellulose deconstruction capability as compared to commercial cellulase preparations. Cotreatment refers to mechanical disruption of lignocellulose during fermentation in order to increase the accessibility of lignocellulose to biological attack and is a promising alternative to thermochemical pretreatment. Consolidated bioprocessing with cotreatment (also referred to as C-CBP) can substantially reduce the cost.

In some embodiments, a method for converting a sugar-rich lignocellulosic feedstock to ethanol or other desired fermentation products is disclosed. The method includes (a) milling the sugar-rich feedstock to produce a mixture containing sugar-rich liquid and lignocellulosic-rich solids; and b) co-fermenting the mixture containing sugar-rich liquid and lignocellulosic-rich solids with a microbial culture, wherein co-fermenting denotes simultaneously fermenting sugar-rich liquid and lignocellulose-rich solids in the same vessel(s) without first separating said liquid and solid; wherein co-fermentation produces ethanol or other desired fermentation products. In some embodiments, no purified enzyme is added in step (a) or (b). In some embodiments, no enzyme that is not produced by the microbial culture is added in step (a) or (b).

In some embodiments, the microbial culture contains one or more living microorganisms. In one aspect, the living microorganisms may be thermophilic anaerobes (C. thermocellum and the hemicellulose-fermenting T. saccharolyticum). In some embodiments, the thermophilic anaerobes are readily able to ferment in the presence of high-intensity milling, whereas more conventional ethanol-producing microbes (yeast, Zymomonas mobilis) are not. In one aspect, the microorganisms disclosed herein may include pure culture or co-culture of microorganisms such as Clostridium thermocellum, Clostridium claraflavum, Caldicellusiruptor bescii, Thermoanaerobacterium saccharolyticum, Thermoanaerobacterium thermosaccharolyticum, or combination thereof. By way of example, strains that may be used in the microbial system include but are not limited to Clostridium thermocellum DSM1313, Clostridium thermocellum ATCC 27405, Clostridium claraflavum 42A, Clostridium claraflavum DSM19732, among others. In another aspect, the microorganism does not include yeast, or Zymomonas mobilis.

In some embodiments, the sugar-rich lignocellulosic feedstock is selected from the group consisting of sugarcane, sugar beets and energy cane. In one aspect, the sugar-rich lignocellulosic feedstock is whole sugar cane.

In some embodiments, step (b) includes a co-treatment step in which mechanical disruption of lignocellulosic-rich solids is performed along with co-fermentation of the mixture containing sugar-rich liquid and lignocellulosic-rich solids. In one aspect, the mechanical disruption is by ball milling, disc milling, or roller milling.

Cotreatment refers to mechanical disruption of lignocellulose during fermentation in order to increase the accessibility of lignocellulose to biological attack and is a promising alternative to thermochemical pretreatment. Co-treatment may be achieved by a number of different ways. By way of example, the disruption may be accomplished by using solid particles (e.g. metal spheres) with density higher than water in the reactor (or fermentor). In another aspect, the disruption may be accomplished by exposure to sheer by intense mixing, passage through an orifice or nozzle, or both. In another aspect, the disruption may be accomplished by pressure cycling, which may lead to formation of bubbles within cellulose particles due to supersaturated CO₂. In another aspect, the disruption may be accomplished by sending lignocellulosic particles through a mill (e.g. disc refiner) outside of the fermentor and recycling them back to the fermentor after the mechanical processing. In another aspect, the means for mechanically disruption and the powering means may include a nozzle or a hydrocyclone with dense beads.

In some embodiments, the living microorganism is capable of surviving the mechanical disruption. In some embodiments, the mechanical disruption is performed under such conditions that the mechanical disruption does not substantially slow the metabolism of the microorganism.

In some embodiments, the co-fermentation of step (b) is performed at a temperature of between 40-70 C, or 50-60 C, or at about 55 C.

In some embodiments, the mixture produced in step (a) is saturated with CO₂.

In some embodiments, the mixture produced in step (a) has a carbohydrate concentration of 50-150 g/L (weight/volume), or 50-100 g/L, or 100 g/L.

microorganisms are pure culture or co-culture of microorganisms selected from the group consisting of Clostridium thermocellum, Clostridium claraflavum, Caldicellusiruptor bescii, Thermoanaerobacterium saccharolyticum, Thermoanaerobacterium thermosaccharolyticum, and combination thereof In some embodiments, the microorganisms are pure culture or co-culture of microorganisms selected from the group consisting of Clostridium thermocellum, Clostridium claraflavum, Caldicellusiruptor bescii, Thermoanaerobacterium saccharolyticum, Thermoanaerobacterium thermosaccharolyticum, and combination thereof.

### BRIEF DESCRIPTION OF THE DRAWINGS

FIG. 1 is a flow chart comparing the conventional process and the process of the present disclosure for converting sugar cane to ethanol.

### DETAILED DESCRIPTION

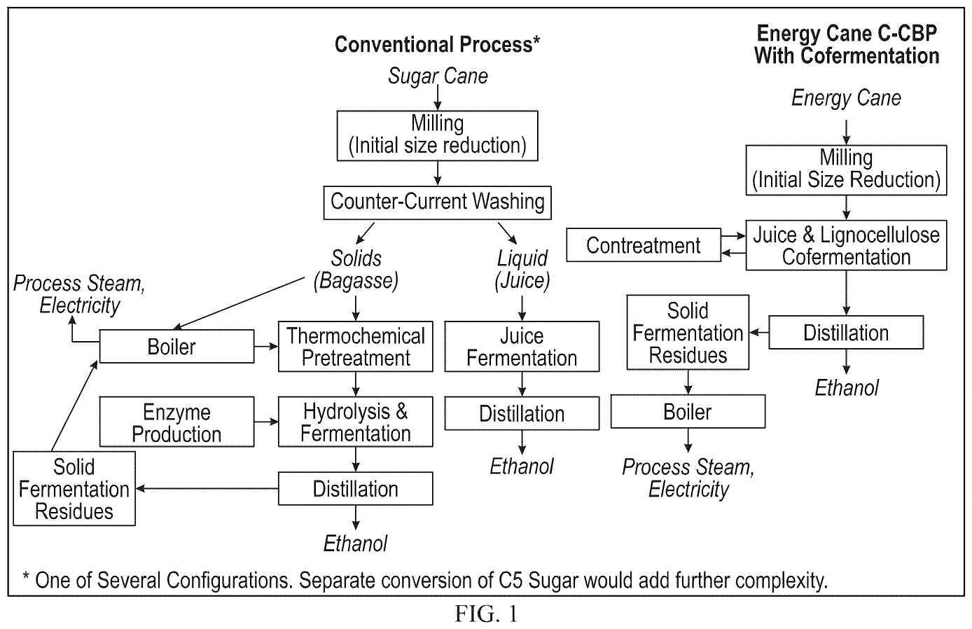

The present disclosure relates to methods for co-fermenting sugar-rich liquids and lignocellulose-rich solids to produce ethanol or other desired fermentation products. FIG. 1 shows a flow chart comparing the conventional process and the process of the present disclosure for converting sugar cane to ethanol. It may be recognized that the conventional process may or may not include pretreatment, hydrolysis, and fermentation of bagasse. It may be recognized that the present disclosure may be applied to other sugar-rich, lignocellulosic feedstocks (e.g., sugar beets, energy cane) instead of sugar cane, and that fermentation could be used to produce molecules other than ethanol. The method of the present disclosure also avoids counter-current washing as well as thermochemical pretreatment, and involves fewer steps compared to the conventional process. Thermochemically pretreating cellulosic biomass that contains substantial quantities of soluble sugars requires the separation of the soluble sugars from the solids, which is expensive. Thus, the elimination of these notably costly and operationally difficult steps has potential for large cost savings.

Embodiments of the present disclosure are further illustrated by the following items.
Item 1. A method for converting a sugar-rich lignocellulosic feedstock to ethanol or other desired fermentation products, comprising:
a) milling said sugar-rich feedstock to produce a mixture containing sugar-rich liquid and lignocellulosic-rich solids; and
b) co-fermenting the mixture containing sugar-rich liquid and lignocellulosic-rich solids with a microbial culture, wherein co-fermenting denotes simultaneously fermenting sugar-rich liquid and lignocellulose-rich solids in the same vessel(s) without first separating said liquid and solid; wherein co-fermentation produces ethanol or other desired fermentation products.
Item 2. The method of Item 1, wherein the sugar-rich lignocellulosic feedstock is selected from the group consisting of sugarcane, sugar beets and energy cane.
Item 3. The method of any preceding items, wherein the sugar-rich lignocellulosic feedstock is whole sugar cane.
Item 4. The method of any preceding items, wherein step (b) includes a co-treatment step in which mechanical disruption of lignocellulosic-rich solids is performed along with co-fermentation of the mixture containing sugar-rich liquid and lignocellulosic-rich solids.
Item 5. The method of any preceding items, wherein the mechanical disruption is by ball milling, disc milling, or roller milling.
Item 6. The method of any preceding items, wherein the microbial culture comprises one or more living microorganisms.
Item 7. The method of any preceding items, wherein the living microorganism is capable of surviving said mechanical disruption.
Item 8. The method of any preceding items, wherein the mechanical disruption is performed under such conditions that the mechanical disruption does not substantially slow the metabolism of the microorganism.
Item 9. The method of any preceding items, wherein the living microorganism is thermophilic, lignocellulose-fermenting anaerobic bacterium or coculture thereof.
Item 10. The method of any preceding items, wherein no purified enzyme is added in step (a) or (b).
Item 11. The method of any preceding items, wherein the co-fermentation of step (b) is performed at a temperature of between 50-60 C.
Item 12. The method of any preceding items, wherein the co-fermentation of step (b) is performed at a temperature of about 55 C.
Item 13. The method of any preceding items, wherein the mixture produced in step (a) is saturated with CO₂.
Item 14. The method of any preceding items, wherein the mixture produced in step (a) has a carbohydrate concentration of 50-100 g/L.
Item 15. The method of any preceding items, wherein the mixture produced in step (a) has a carbohydrate concentration of 100 g/L.
Item 16. The method of any preceding items, wherein the microorganisms are pure culture or co-culture of microorganisms selected from the group consisting of Clostridium thermocellum, Clostridium claraflavum, Caldicellusiruptor bescii, Thermoanaerobacterium saccharolyticum, Thermoanaerobacterium thermosaccharolyticum, and combination thereof.
Item 17. The method of any preceding items, wherein the microorganism does not include yeast, or Zymomonas mobilis.

It will be readily apparent to those skilled in the art that the systems and methods described herein may be modified and substitutions may be made using suitable equivalents without departing from the scope of the embodiments disclosed herein. Having now described certain embodiments in detail, the same will be more clearly understood by reference to the following examples, which are included for purposes of illustration only and are not intended to be limiting.

The contents of all cited references (including literature references, patents, patent applications, and websites) that may be cited throughout this application or listed below are hereby expressly incorporated by reference in their entirety for any purpose into the present disclosure. The disclosure may employ, unless otherwise indicated, conventional techniques of microbiology, molecular biology and cell biology, which are well known in the art.

The disclosed methods may be modified without departing from the scope hereof. It should be noted that the matter contained in the above description or shown in the accompanying drawings should be interpreted as illustrative and not in a limiting sense.

What is claimed is:

1. A method for converting a sugar-rich lignocellulosic feedstock to ethanol or other desired fermentation products, comprising:
a) milling said sugar-rich feedstock to produce a mixture containing sugar-rich liquid and lignocellulosic-rich solids; and
b) co-fermenting the mixture containing sugar-rich liquid and lignocellulosic-rich solids with a microbial culture, wherein co-fermenting denotes simultaneously fermenting sugar-rich liquid and lignocellulose-rich solids in the same vessel(s) without first separating said liquid and solid; wherein co-fermentation produces ethanol or other desired fermentation products.

2. The method of claim 1, wherein the sugar-rich lignocellulosic feedstock is selected from the group consisting of sugarcane, sugar beets and energy cane.

3. The method of claim 1, wherein the sugar-rich lignocellulosic feedstock is whole sugar cane.

4. The method of claim 1, wherein step (b) includes a co-treatment step in which mechanical disruption of lignocellulosic-rich solids is performed along with co-fermentation of the mixture containing sugar-rich liquid and lignocellulosic-rich solids.

5. The method of claim 4, wherein the mechanical disruption is by ball milling, disc milling, or roller milling.

6. The method of claim 1, wherein the microbial culture comprises one or more living microorganisms.

7. The method of claim 4, wherein the living microorganism is capable of surviving said mechanical disruption.

8. The method of claim 4, wherein the mechanical disruption is performed under such conditions that the mechanical disruption does not substantially slow the metabolism of the microorganism.

9. The method of claim 2, wherein the living microorganism is thermophilic, lignocellulose-fermenting anaerobic bacterium or coculture thereof.

10. The method of claim 2, wherein no purified enzyme is added in step (a) or (b).

11. The method of claim 2, wherein the co-fermentation of step (b) is performed at a temperature of between 50-60 C.

12. The method of claim 2, wherein the co-fermentation of step (b) is performed at a temperature of about 55 C.

13. The method of claim 1, wherein the mixture produced in step (a) is saturated with CO₂.

14. The method of claim 1, wherein the mixture produced in step (a) has a carbohydrate concentration of 50-100 g/L.

15. The method of claim 1, wherein the mixture produced in step (a) has a carbohydrate concentration of 100 g/L.

16. The method of claim 9, wherein the microorganisms are pure culture or co-culture of microorganisms selected from the group consisting of Clostridium thermocellum, Clostridium claraflavum, Caldicellusiruptor bescii, Thermoanaerobacterium saccharolyticum, Thermoanaerobacterium thermosaccharolyticum, and combination thereof.

17. The method of claim 9, wherein the microorganism does not include yeast, or Zymomonas mobilis.
